(19) United States
(12) Patent Application Publication
(10) Pub. No.: US 2009/0239881 A1
(43) Pub. Date: Sep. 24, 2009

(54) Combinations of Flibanserin with Caffeine, Process for Their Preparation and Use Thereof as Medicaments

(75) Inventor: Reinhold Becker, Bingen (DE)

Correspondence Address: Michael P. Morris;Boehringer Ingelheim USA Corporation, 900 Ridgebury Road, Ridgefield, CT, 06877-0368, US

(73) Assignee: Boehringer Ingelheim International GmbH, Ingelheim (DE)

(21) Appl. No.: 12/306,946

(22) PCT Filed: Jul. 11, 2007
(86) PCT No.: PCT/EP2007/057065

§ 371 Date: May 28, 2009

Related U.S. Application Data
(60) Provisional application No. 60/830,989, filed on Jul. 14, 2006.

(51) Int. Cl. A61K 31/496

(52) U.S. Cl. 514/254.06

(57) ABSTRACT

The invention is directed to a combination of flibanserin or a pharmaceutically acceptable derivative thereof and caffeine or a pharmaceutically acceptable derivative thereof for the treatment of Sexual Disorders in humans, particularly women.

### FIELD OF THE INVENTION

The invention relates to combinations of flibanserin with caffeine, process for their preparation and use thereof as medicaments.

### BACKGROUND OF THE INVENTION

Sexual Dysfunctions or Disorders, particularly Female Sexual Dysfunctions or Disorders are highly prevalent and it is estimated that about 20 to 50% of the women are affected. Up to now there are no approved pharmacological treatments since clinical development and off-label usage has focused mainly on hormone products. Furthermore, there exists a need for the development of improved therapies which are more individualized and adapted to the problems of each individual case.

The compound 1-[2-(4-(3-trifluoromethyl-phenyl)piperazin-1-yl)ethyl]-2,3-dihydro-1H-benzimidazol-2-one (flibanserin) is disclosed in form of its hydrochloride in European Patent Application EP-A-526434 and has the following chemical structure:

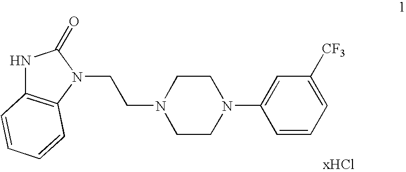

Flibanserin shows affinity for the 5-HT_{1A} and 5-HT₂-receptor. It is therefore a promising therapeutic agent for the treatment of a variety of diseases, for instance depression, schizophrenia, and anxiety.

According to WO 03/035072 A1 it is known to use flibanserin in the treatment of Sexual Disorders; in WO 2005/102343 A1 it is described to use flibanserin in the treatment of premenstrual and other female Sexual Disorders.

However, the known use of flibanserin has in the disadvantage that sometimes mild or moderate symptoms such as sedation may occur.

Therefore, it is an object of the present invention to overcome the above-mentioned deficiencies and to attenuate or minimize the negative symptoms which may occur with the treatment of flibanserin.

### DESCRIPTION OF THE INVENTION

Surprisingly, it has been found that it is possible to combine flibanserin with caffeine in order to significantly reduce the described side-effects to a minimum and at the same time increasing the efficacy of the treatment.

It is therefore provided a combination of flibanserin or a pharmaceutically acceptable derivative thereof and caffeine or a pharmaceutically acceptable derivative thereof for the treatment of Sexual Disorders in humans, particularly women.

It has surprisingly been found that a combination of flibanserin/caffeine or derivatives thereof act synergistically in humans, particularly women, or show cooperative effects of the individual active ingredients. The combination of both ingredients is superior to the effectiveness of the single active substance flibanserin and may result in a dose reduction and reduces side-effects to a minimum.

Flibanserin can be used in form of the free base or in form of any known pharmaceutically acceptable derivative thereof such as its pharmaceutically acceptable acid addition salts and/or optionally in form of the hydrates and/or solvates thereof. Suitable acid addition salts include for example those of the acids selected from succinic acid, hydrobromic acid, acetic acid, fumaric acid, maleic acid, methanesulphonic acid, lactic acid, phosphoric acid, hydrochloric acid, sulphuric acid, tartaric acid and citric acid. Mixtures of the above-mentioned acid addition salts may also be used. From the aforementioned acid addition salts the hydrochloride and the hydrobromide, particularly the hydrochloride, are preferred.

If flibanserin is used in form of the free base, it is preferably used in form of flibanserin polymorph A which represents the free base of flibanserin in a specific polymorphic form. Polymorph A and a process for its preparation are disclosed in WO 03/014079 A1, the whole disclosure thereof being incorporated by reference into the present specification.

Pure caffeine C₈H₁₀N₄O₂ is a white odourless crystalline powder and is a very weak base which may be dissolved in water and organic solvents such as ethanol. Pharmaceutically acceptable derivatives of caffeine are for example acid salts thereof, e.g. caffeine sodium salicylate. It may be used in any suitable form according to the intended application form.

The type of combining the two components, flibanserin or a derivative thereof and caffeine or a derivative thereof, is not limited according to the present invention. Any possible kind of administration or timing is possible insofar the combined effects of flibanserin and caffeine are achieved in the patient to be treated. As a result, both components may be administered simultaneously or separately, in a timely coordinated manner.

If using caffeine in a separate dosage form or unit it should be noted that caffeine has a relatively short action time. Usually caffeine is completely absorbed by the stomach and small intestine within 45 minutes of ingestion. After ingestion, caffeine has a physiological half-life of three and a half to six hours. It is widely distributed in total body water and is eliminated by apparent first-order kinetics. Therefore, caffeine or derivatives thereof should be administered directly prior to or after the administration of flibanserin or derivatives thereof. In order to simplify the therapy, combination preparations, particularly combination preparations having fixed doses, may be used.

According to the present invention it has been found that caffeine enhances the potency of flibanserin in some cases up to a synergistic effectivity. Not being bound by any theory it is assumed that caffeine which is a xanthine alkaloide develops its central nervous system (CNS) stimulating capability to an enhanced extent when used in combination with flibanserin and significantly improves the spectrum of activity thereof.

According to a further aspect of the present invention it is provided a combination of a pharmaceutically effective amount of flibanserin or a pharmaceutically acceptable derivative thereof and a pharmaceutically effective amount of caffeine or a pharmaceutically acceptable derivative thereof for the treatment of Sexual Disorders in humans, particularly women.

The dose range of flibanserin or a pharmaceutically acceptable derivative thereof applicable per day is usually from 0.1 to 400 mg, preferably from 1.0 to 300 mg, more preferably from 2 to 200 mg, most preferably 25, 50 or 100 mg. Each dosage unit may conveniently contain from 0.01 to 100 mg, preferably 0.1 to 50 mg.

The dose range of caffeine or a pharmaceutically acceptable derivative thereof applicable per day is usually from 0.1 to 400 mg, preferably from 1.0 to 300 mg, more preferably from 2 to 200 mg. Each dosage unit may conveniently contain from 0.01 to 100 mg, preferably 0.1 to 50 mg.

It is also possible to use commercially available caffeine containing products, for example caffeine tablets usually having a caffeine content of 100 to 300 mg.

The actual pharmaceutically effective amount or therapeutic dosage will of course depend on factors known by those skilled in the art such as age and weight of the patient, route of administration and severity of disease. In any case the combination will be administered at dosages and in a manner which allows a pharmaceutically effective amount to be delivered based upon patient's unique condition.

The combination according to the present invention may be provided simultaneously in one and the same dosage form, i.e. in form of a combination preparation, for example the two components may be incorporated in one tablet, e.g. in different layers of said tablet. The combination may be also provided separately, in form of a free combination, i.e. flibanserin or a pharmaceutically acceptable derivative thereof is provided in one dosage form and caffeine or a pharmaceutically acceptable derivative thereof is provided in another dosage form. These two dosage forms may be equal dosage forms, for example a co-administration of two tablets, one containing a therapeutically effective amount of caffeine and one containing a therapeutically effective amount of flibanserin. It is also possible to combine different administration forms, if desired. Any type of suitable administration forms may be provided. The administration forms are not limited according to the present invention. Particularly preferred administration routes are oral, rectal, parenteral administration or nasal inhalation.

The dosage or administration forms are not limited, in the frame of the present invention any suitable dosage form may be used. Exemplarily the dosage forms may be selected from solid preparations such as tablets, capsules, pills, pellets, dragees, powders, troches, suppositories, liquid preparations such as solutions, suspensions, emulsions, drops, syrups, elixirs, or gaseous preparations such as aerosols, sprays and the like.

The dosage forms are advantageously formulated in dosage units, each dosage unit being adapted to supply a single dose of each active component being present. Depending from the administration route and dosage form the ingredients are selected accordingly.

The dosage forms are administered to the patient 1, 2, 3, or 4 times daily. It is preferred that the compounds of the invention be administered either three or fewer times, more preferably once or twice daily.

More preferably, the dose is administered to a patient in the morning and the evening, more preferably once in the morning and once in the evening, most preferably once in the evening only.

As a matter of course, beside the active ingredient(s), such as flibanserin and caffeine, additives, excipients, carriers, technological adjuvants suitable in pharmaceutical formulations may be present in the dosage forms selected such as binders, lubricants, glidants, agents to improve flowability, granulating agents, anti-caking agents, agglomeration inhibitors, pore formers, anti-adherents, anti-tacking agent, anti-sticking agent, flavours, aromatiziers, dyes or colorants, preservatives, plasticizers, diluents, wetting agents, sweeteners, disintegrants, tonicity agents, chelating agents, stabilizers, solubilizers, antioxidants, fillers, pigments and the like. These pharmaceutically acceptable formulating agents are e.g. present in order to promote the manufacture, compressibility, appearance and/or taste of the preparation. Other conventional additives known in the art can also be included. The above listing is not intended to be of limitative character, the skilled person is familiar with further examples.

It is deemed to be within the capabilities of the skilled person, and the existing literature about dosage forms and usual components can be consulted in order to arrive at the constitution, size and appearance of a desired dosage form. Subject of the present invention is also a process for manufacturing a combination wherein flibanserin or a pharmaceutically acceptable derivative thereof and caffeine or a pharmaceutically acceptable derivative thereof in addition with suitable additive(s), excipient(s), carrier(s), and adjuvant(s) are brought into a suitable dosage form.

In studies of female patients suffering from Sexual Disorders it has been found that flibanserin, optionally in form of the free base, as well as a pharmaceutically acceptable derivative such as the pharmaceutically acceptable acid addition salts and/or optionally the hydrates and/or solvates thereof has a positive effect on Sexual Disorders and/or Dysfunctions for example it shows sexual desire enhancing properties. The combination with caffeine or derivatives thereof improves the efficacy of said active substance.

Therefore the present invention is also directed to the use of a combination of flibanserin or a pharmaceutically acceptable derivative thereof and caffeine or a pharmaceutically acceptable derivative thereof for the preparation of a medicament in case of a pharmaceutical combination preparation, or two medicaments in case of a free combination, for the treatment of Sexual Disorder in humans, particularly women.

The generic term “Sexual Disorders” or “Sexual Dysfunctions” according to the present invention shall be understood within its broadest meaning and shall include all kind of Sexual Disorders and Dysfunctions known. “Sexual disorders” or “Sexual Dysfunctions”, both expressions being used virtually synonymously in the present invention and may be characterized by a disturbance in sexual desire, in the physiological changes that characterize the sexual response cycle or by pain associated with sexual intercourse. The sexual response cycle may be divided in the phases Desire, Excitement, Orgasm and Resolution and the disorders of sexual response may occur at one or more of these phases, multiple disorders or dysfunctions may be present. Sexual disorders may cause distress and personal difficulty and may be associated with other disorders such as mood disorders or anxiety disorders (Obsessive-Compulsive Disorder, Panic Disorder with Agoraphobia and specific Phobia) (see Diagnostic and Statistical Manual of Mental Disorders, 4th edition, Text Revision. Washington D.C., American Psychiatric Association, 2000).

Sexual Disorders and/or Sexual Dysfunctions (hereinafter simply referred to as “Sexual Disorder(s)” or “Disorder(s)”) are categorized into several main types which may be further divided in several subtypes all of which are included herein.

Examples of Sexual Disorders are Sexual Desire Disorders, (i.e., Hypoactive Sexual Desire Disorder, Sexual Aversion Disorder), Sexual Arousal Disorders (i.e., Female Sexual Arousal Disorder, Male Erectile Disorder), Orgasmic Disorders (i.e., Female Orgasmic Disorder, Male Orgasmic Disorder, Premature Ejaculation). Sexual Pain Disorders (Dyspareunia, Vaginismus, Noncoital Pain Disorder), Sexual Dysfunction due to a General Medical Condition, Substance-Induced Sexual Dysfunction, and Sexual Dysfunction not otherwise specified (cf. Diagnostic and Statistical Manual of Mental Disorders, ibid.).

According to Diagnostic and Statistical Manual of Mental Disorders, 4th edition, Text Revision, Washington D.C., American Psychiatric Association, 2000, the disclosure thereof being incorporated in the present specification by reference, Hypoactive Sexual Desire Disorder (HSDD) is characterized by a general loss of sexual desire leading to distress. HSDD may be in detail defined to be a deficiency or absence of sexual fantasies and desire for sexual activity (criterion A) whereby the dysfunction must cause marked distress or interpersonal difficulties (criterion B) and the Sexual Disorder is not better accounted for by another disorder (criterion C).

Sexual Aversion Disorder is defined as a persistent or recurrent extreme aversion to, and avoidance of, all or almost all genital sexual contact with a sexual partner. Sexual Arousal Disorder is characterized to be a persistent or recurrent inability to attain, or maintain until completion of the sexual activity. Orgasmic Disorders is a persistent or recurrent delay in, or absence of, orgasm following normal sexual excitement phase. Sexual Pain Disorders is related with genital pain which may be associated with sexual intercourse or the involuntary contraction of the perineal muscles surrounding the outer third of the vagina.

Sexual Dysfunction due to a General Medical Condition may be determined based on history, laboratory findings or physical examination that the Sexual Disorder is fully explained by direct physiological effects of a general medical condition.

Further, Substance-Induced Sexual Dysfunction may be a disorder or dysfunction exclusively caused by the physiological effects of a drug abuse, a medication or toxin exposure. It depends on the type or amount of the substance used or the duration of use or exposure.

The Sexual Dysfunction not otherwise specified includes Sexual Dysfunctions that do not meet criteria for any other specific Sexual Dysfunction.

It should be noted that the definitions developed for the categorization of Sexual Disorders have no fixed or exact limits, but transitions and/or overlappings may be possible. One type of Sexual Disorder may also occur in association with other Sexual Disorders or Sexual Dysfunctions. Then, for example, the predominant Sexual Disorder is selected, the type of disorder assigned is not better accounted for by another type of disorder.

The subtypes in order to further categorize Sexual Disorder indicate the onset (lifelong type and acquired type), context (generalized type and situational type) and etiological factors (due to psychological factors and due to combined factors) associated with the Sexual Disorder. These subtypes do not apply to a diagnosis of Sexual Dysfunction due to a general medical condition or Substance-induced Sexual Dysfunction.

The “lifelong type” refers to such Sexual Disorders of the present invention, which have been present since the onset of sexual functioning. The “acquired type” applies to such Sexual Disorders of the present invention which developed only after a period of normal sexual functioning. The “generalized type” refers to such Sexual Disorders of the present invention wherein the disorder is not limited to certain types of stimulation, situations, or partners. The “situational type” applies to such Sexual Disorders of the present invention wherein the disorder is limited to certain types of stimulation, situations, or partners. The subtype due to “psychological factors” applies when psychological factors are judged to have the major role in the onset, severity, exacerbation, or maintenance of the Sexual Disorder, and general medical conditions and substance play no role in the etiology of the Sexual Disorder. Finally the subtype due to “combined factors” applies when 1) psychological factors are judged to have a role in the onset, severity, exacerbation, or maintenance of the Sexual Disorder, and 2) a general medical condition or substance use is also judged to be contributory but is not sufficient to account for a Sexual Disorder (cf. Diagnostic and Statistical Manual of Mental Disorders, 4th edition, Text Revision. Washington D.C., American Psychiatric Association, 2000).

Furthermore, Sexual Disorders and/or Dysfunctions may be also treated in female patients being in pre-menopausal or post-menopausal status. In other words the above mentioned types of Sexual Disorders may be also treated in pre-menopausal or post-menopausal women. Also premenstrual disorders should be included in Sexual Disorders, for example Premenstrual Disorders selected from the group consisting of Premenstrual Dysphoria, Premenstrual Syndrome, Premenstrual Dysphoric Disorder. Premenstrual and other Sexual Disorders are described in WO 2005/102343 the whole disclosure thereof being incorporated into the present specification by reference.

Sexual Disorders of main interest are for example Sexual Desire Disorders, (such as Hypoactive Sexual Desire Disorder (HSDD), Sexual Aversion Disorder), loss of sexual desire, lack of sexual desire, decreased sexual desire, inhibited sexual desire, loss of libido, libido disturbance, and frigidity, Orgasmic Disorders, Sexual Pain Disorders, Sexual Dysfunction due to a General Medical Condition, Substance-Induced Sexual Dysfunction, and Sexual Dysfunction not otherwise specified; particularly HSDD.

Disorders of sexual desire are, for example, described in WO 03/035072 A1, the whole disclosure thereof being incorporated into the present specification by reference.

The beneficial effects of a combination of flibanserin and caffeine can be observed in all kind of Sexual Disorders of patients, particularly female patients, independent of the main type or subtype, independent of the onset, context and etiological factors associated with the Sexual Disorder. That is regardless of whether the Sexual Disorder existed lifelong or was acquired, or independent of context and etiologic origin, the treatment will be effective.

The Advantages of the Present Invention are Manifold:

The inventive combination of flibanserin and caffeine or their derivatives acts synergistically in humans, particularly women, or shows cooperative effects of the individual components. The combination of both ingredients is superior to the effectiveness of the single active substance flibanserin.

The combination according to the present invention makes it possible to reduce the dose to the minimum dose necessary to obtain a therapeutically positive effect in the sense of a meaningful therapeutic response.

The therapy offers reduced side effects due to the optimized medication. As a result side-effects such as sedation are of lesser significance. The use according to the present invention achieves improved therapeutic effects adapted to the specific needs of a patient.

The beneficial effects of the combination flibanserin/caffeine or pharmaceutically acceptable derivatives thereof can be observed in all kind of Sexual Disorders of patients, particularly female patients, regardless of whether the Sexual Disorder existed lifelong or was acquired, or independent of context and etiologic origin.

1. A pharmaceutical composition comprising:
(a) flibanserin or a pharmaceutically acceptable derivative thereof; and
(b) caffeine or a pharmaceutically acceptable derivative thereof.

2. The pharmaceutical composition according to claim 1, comprising:
(a) a pharmaceutically effective amount of flibanserin or a pharmaceutically acceptable derivative thereof; and
(b) a pharmaceutically effective amount of caffeine or a pharmaceutically acceptable derivative thereof.

3. A kit comprising:
(a) a first pharmaceutical composition comprising flibanserin or a pharmaceutically acceptable derivative thereof; and
(b) a second pharmaceutical composition comprising caffeine or a pharmaceutically acceptable derivative thereof.

4. The kit according to claim 3, comprising:
(a) a first pharmaceutical composition comprising a pharmaceutically effective amount of flibanserin or a pharmaceutically acceptable derivative thereof; and
(b) a second pharmaceutical composition comprising a pharmaceutically effective amount of caffeine or a pharmaceutically acceptable derivative thereof.

5. The pharmaceutical composition according to claim 1, wherein the amount of flibanserin or the pharmaceutically acceptable derivative thereof ranges from 0.01 to 400 mg.

6. The pharmaceutical composition according to claim 1, wherein the amount of caffeine or the pharmaceutically acceptable derivative thereof ranges from 0.01 to 400 mg.

7. The pharmaceutical composition according to claim 1, wherein the flibanserin is polymorph A of flibanserin.

8. The pharmaceutical composition according to claim 1, wherein the pharmaceutically acceptable derivative of flibanserin is an acid addition salt, hydrate, or solvates thereof.

9. The pharmaceutical composition according to claim 1, wherein the pharmaceutically acceptable acid addition salt is selected from the salts formed by the acids selected from succinic acid, hydrobromic acid, acetic acid, fumaric acid, maleic acid, methanesulphonic acid, lactic acid, phosphoric acid, hydrochloric acid, sulphuric acid, tartaric acid, citric acid, and mixtures thereof.

10. The pharmaceutical composition according to claim 1, wherein the pharmaceutically acceptable derivative of caffeine is an acid salt thereof.

11. The pharmaceutical composition according to claim 1, wherein the pharmaceutical composition is suitable for oral, rectal, or parenteral administration, or for nasal inhalation.

12. The pharmaceutical composition according to claim 1 further comprising suitable additive(s), excipient(s), carrier(s), or adjuvant(s).

13.-19. (canceled)
